(19) United States
(12) Patent Application Publication
(10) Pub. No.: US 2010/0310654 A1
(43) Pub. Date: Dec. 9, 2010

(54) HOMEOPATHIC REMEDIES FOR USE BEFORE AND AFTER AESTHETIC INJECTABLE AND SURGICAL PROCEDURES TO MINIMIZE SWELLING AND BRUISING

(76) Inventor: Andrew A. Jacono, Great Neck, NY (US)

Correspondence Address: THE FARRELL LAW FIRM, LLP, 290 Broadhollow Road, Suite 210E, Melville, NY, 11747, US

(21) Appl. No.: 12/745,427

(22) PCT Filed: Dec. 1, 2008
(86) PCT No.: PCT/US08/85149

§ 371 Date: May 28, 2010

Related U.S. Application Data
(60) Provisional application No. 60/991,389, filed on Nov. 30, 2007.

(51) Int. Cl. A61K 9/28; A61K 38/48; A61P 17/00; A61P 17/02

(52) U.S. Cl. 424/474; 424/94.65

(57) ABSTRACT

The present invention generally relates to all-in-one homeopathic remedies for use before and after aesthetic injectable treatment and/or surgical procedures to help minimize swelling and bruising.

### PRIORITY

This application claims priority to U.S. provisional patent application No. 60/991,389 filed Nov. 30, 2007, the contents of which are incorporated herein by reference.

### FIELD OF THE INVENTION

The present invention generally relates to homeopathic remedies that help optimize healing after aesthetic injectable and surgical procedures.

### BACKGROUND OF THE INVENTION

Physicians are continuously looking for ways to improve and expedite healing following aesthetic injectable and surgical procedures. Physicians try various techniques and pharmaceutical companies invariably test tissue glues and systemic agents in an attempt to find combinations that will result in a more rapid recovery. Additionally, patients try home remedies, and homeopathic therapies for more rapid healing. To date, however, such attempts to promote rapid recovery have been largely unsuccessful.

It is an object of the invention to provide a homeopathic remedy that helps optimize and promote rapid healing after aesthetic injectable and surgical procedures.

### SUMMARY OF THE INVENTION

The present invention relates to a novel homeopathic remedy that assists in minimizing swelling and bruising after aesthetic injections. In a preferred embodiment, the invention provides a homeopathic remedy that optimizes and promotes rapid healing, to reduce bruising, swelling and discomfort following surgery.

### DETAILED DESCRIPTION OF THE INVENTION

The claimed invention relates to all-in-one homeopathic remedies for use before and after aesthetic injectable treatment and/or surgical procedures to help minimize swelling and bruising.

Homeopathy is a system of therapy that focuses on health and wholeness rather than disease. This science was founded in the 18^{th} century and is based on teachings of Hippocrates regarding the Law of Similars, or similia similibus curentur, i.e., like is cured by like. Simply stated, any substance that can cause symptoms when taken at higher doses can also cure those very same symptoms in an unhealthy person when taken in very small dose.

Scientific studies indicate that homeopathic remedies like Arnica Montana and dietary supplements like Bromelaine and Hyaluronic Acid can help minimize swelling and bruising and speed healing. In fact, medical experts have been treating patients with homeopathy for centuries. Today, with aesthetic injectables and facial plastic surgery on the rise, the need is evident for an easy to use system that provides an all-in-one, convenient solution featuring precise doses of the most refined, concentrated, bio available formulas. The claimed invention provides just such an all-in-one homeopathic system referred to herein as the J PAK SYSTEM. J PAK SYSTEMS combine the most refined, concentrated, bio available formulas in convenient, one dose packets specifically for use before and after aesthetic injections. In one embodiment, the J PAK systems are in tablet form, although the invention contemplates other means of administering including, but not limited to liquids, gels, and the like.

The selected remedy should closely match the symptoms. In conditions where self-treatment is appropriate, unless otherwise directed by a physician, a lower potency (6X, 6C, 12X, 12C, 30X, or 30C) should be used. Many, new to homeopathy, mistake potency or dilution with strength. There is actually no correlation between potency and the strength of a homeopathic medicine. A homeopathic medicine at 30C potency is not stronger than the same medicine at 6C or 3C. The difference is in their action. While a 6C potency is better suited for a local symptom, a 30C or higher potency is more appropriate for general conditions such as allergy, stress or sleep disorders.

Homeopathic medicine potency in the U.S.A. is described by a two part, number and letter designation. Potencies are referred to as 12X, 6C, 30C, 10M or 6LM, with the number part indicating a number of times that a source substance has been homeopathically diluted and the Letter portion indicating a dilution rate. X, being the Roman numeral 10, is the slowest dilution rate of 1/10 and LM is the fastest dilution rate of 1/50,000.

In one embodiment, the invention discloses a homeopathic system for use before and after aesthetic injections to help minimize swelling and bruising. This system, referred to as J PAK SYSTEM No. 1, includes:
High Potency Arnica Montana, a perennial herb, indigenous to Central Europe that reduces post-traumatic bruising and swelling. The high potency Arnica Montana utilized is 1,000 times more potent than what is typically commercially available to deliver optimal results.
Bromelain, an enzyme derived from pineapple stems to reduce edema (swelling) and ecchymosis (bruising) following surgical and non-surgical trauma to the face. The Bromelain employed is enterically coated so it is absorbed by the body and not digested

A typical J PAK SYSTEM 1 serving size delivers approximately 1M HPUS Arnica Montana and 400 mg Bromelain (6000 PU/mg). The letters HPUS indicate that the components in this product are officially monographed in the Homeopathic Pharmacoepia of the United States. Optional ingredients such as calcium can also be included, provided that they do not have a detrimental affect on the remedies effectiveness.

In one embodiment, from 50-150 mg calcium is included, in another embodiment from 60-100 mg, and in yet another embodiment, from 70-85 mg calcium is included per tablet. A typical tablet contains approximately 78 mg calcium as di-calcium phosphate.

In another embodiment, the above amounts can be varied up to +/−20% from the above stated values.

J PAK SYSTEM No. 1 is typically supplied in a five-day supply of 10 packets containing one tablet per packet (one serving), but as one of ordinary skill will recognize, this can be varied in accordance with the needs of the patient.

In another embodiment, the invention discloses a homeopathic system for use to optimize and promote rapid healing and minimize swelling, bruising and discomfort following surgery. This system, referred to as J PAK SYSTEM No. 2, includes:
Arnica Montana—as stated above
Bromelain—as stated above
Hyaluronic Acid, a carbohydrate component of the extracellular (outside the cells) matrix of skin. It is produced by fibroblasts (cells in the skin) during wound repair and helps accelerate healing.
Glucosamine compounds have several beneficial effects on the skin and its cells. Because it stimulates Hyaluronic Acid synthesis, it also can accelerate wound healing, improve skin hydration and decrease wrinkles.
Vitamin C, an essential cofactor for collagen production. Wound healing requires the production of new collagen.
Zinc, an essential trace element in the human body. It serves as a cofactor in skin cell migration during wound repair.

J PAK SYSTEM No. 2 serving size delivers Arnica Montana and Bromelain in approximately the same amounts as in the J PAK SYSTEM No. 1. A typical serving size (three tablets) also delivers from 750-1500 mg, in another embodiment from 900-1200 mg, and in still another embodiment about 1000 mg Vitamin C; and hyaluronic acid. The hyaluronic acid is typically delivered in tablet form and comprises from 10-20 mg, in another embodiment from 12-18 mg and in still another embodiment approximately 15 mg zinc; from 500-1000 mg, in another embodiment from 600-800 mg, and in another embodiment about 750 mg glucosamine sulfate KCL (or equilivent thereof); from 300-500 mg, in another embodiment from 300-400 mg, and in yet another embodiment about 333 mg BioCell Collagen II per tablet. BioCell Collagen II itself comprises approximately 200 mg, hydrolyzed collagen Type II, approximately 66 mg chondroitin sulfate and approximately 33 mg hyaluronic acid. The above amounts can be varied by up to 20%.

J PAK SYSTEM No. 2 is typically supplied in a 14 day supply of 28 packets, but as one of ordinary skill will recognize, this can be varied in accordance with the specific needs of the patient.

While the invention has been shown and described with reference to certain exemplary embodiments of the present invention thereof, it will be understood by those skilled in the art that various changes in form and details may be made therein without departing from the spirit and scope of the present invention as defined by the appended claims and equivalents thereof.

What is claimed:

1. A homeopathic system for minimizing swelling and bruising after aesthetic injections, said system comprising high potency Arnica Montana in combination with Bromelain, wherein said Bromelain is enterically coated to be absorbed and not digested.

2. The system of claim 1 wherein a serving size comprises about +/−20% of 1M HPUS Arnica Montana, from about 300-600 mg Bromelain (6000 PU/mg), and optionally from 50-150 mg calcium.

3. The system of claim 2 wherein said serving size comprises approximately 1M HPUS Arnica Montana, approximately 400 mg Bromelain (6000 PU/mg), and optionally from about 70-85 mg calcium.

4. The system of claim 3 wherein said serving size is provided in a single tablet.

5. A method for minimizing swelling and bruising of a patient undergoing aesthetic injections, said method comprising administering to said patient an effective amount of a homeopathic system, said system comprising high potency Arnica Montana in combination with Bromelain, wherein said Bromelain enterically coated so it is absorbed by the body and not digested.

6. The method of claim 5 wherein said effective amount comprises about +/−20% of 1M HPUS Arnica Montana, from about 300-600 mg Bromelain (6000 PU/mg), and optionally from 50-150 mg calcium.

7. The method of claim 6 wherein said effective amount comprises approximately 1M HPUS Arnica Montana, approximately 400 mg Bromelain (6000 PU/mg), and optionally from about 70-85 mg calcium.

8. The method of claim 5 wherein said effective amount is provided in a single tablet.

9. A homeopathic system for use in optimizing and promoting rapid healing and minimizing swelling, bruising and discomfort following surgery, said system comprising a combination of high potency Arnica Montana, Bromelain, wherein said Bromelain enterically coated to be absorbed and not digested, Hyaluronic Acid, Glucosamine, Vitamin C, and Zinc.

10. The system of claim 9 wherein a serving size comprises from about +/−20% of 1M HPUS Arnica Montana, from about 300-600 mg Bromelain (6000 PU/mg), optionally from 50-150 mg calcium, from 750-1500 mg Vitamin C, from 10-20 mg zinc; from 500-1000 mg Glucosamine Sulfate KCL, from 300-500 mg BioCell Collagen II, about 200 mg, hydrolyzed collagen Type II, approximately 66 mg chondroitin sulfate and approximately 33 mg hyaluronic acid.

11. The system of claim 10 wherein said serving size comprises about 1M HPUS Arnica Montana, about 400 mg Bromelain (6000 PU/mg), optionally about 78 mg calcium, about 1000 mg Vitamin C, about 15 mg zinc; about 750 mg Glucosamine Sulfate KCL, about 333 mg BioCell Collagen II, about 200 mg, hydrolyzed collagen Type II, approximately 66 mg chondroitin sulfate and approximately 33 mg hyaluronic acid.

12. The system of claim 11 wherein said serving size is delivered in one packet, and said packet consists of three tablets.

13. A method for optimizing and promoting rapid healing and minimizing swelling, bruising and discomfort following surgery, said method comprising administering to said patient an effective amount of a homeopathic system, said system comprising a combination of high potency Arnica Montana, Bromelain, wherein said Bromelain enterically coated to be absorbed and not digested, Hyaluronic Acid, Glucosamine, Vitamin C, and Zinc.

14. The method of claim 13 wherein an effective amount comprises from about +/−20% of 1M HPUS Arnica Montana, from about 300-600 mg Bromelain (6000 PU/mg), optionally from 50-150 mg calcium, from 750-1500 mg Vitamin C, from 10-20 mg zinc; from 500-1000 mg Glucosamine Sulfate KCL, from 300-500 mg BioCell Collagen II, about 200 mg, hydrolyzed collagen Type II, approximately 66 mg chondroitin sulfate and approximately 33 mg hyaluronic acid.

15. The method of claim 13 wherein said effective amount comprises about 1M HPUS Arnica Montana, about 400 mg Bromelain (6000 PU/mg), optionally about 78 mg calcium, about 1000 mg Vitamin C, about 15 mg zinc; about 750 mg Glucosamine Sulfate KCL, about 333 mg BioCell Collagen II, about 200 mg, hydrolyzed collagen Type II, approximately 66 mg chondroitin sulfate and approximately 33 mg hyaluronic acid.

16. The method of claim 13 wherein said system is provided in one packet comprising three tablets.
